(19) United States
(12) Patent Application Publication
(10) Pub. No.: US 2012/0190984 A1
(43) Pub. Date: Jul. 26, 2012

(54) ULTRASOUND SYSTEM WITH OPACITY SETTING UNIT

(75) Inventors: Sung Yoon KIM, Seoul (KR); Dong Gyu HYUN, Seoul (KR); Jong Sik KIM, Seoul (KR)

(73) Assignee: SAMSUNG MEDISON CO., LTD.

(21) Appl. No.: 13/358,961

(22) Filed: Jan. 26, 2012

(30) Foreign Application Priority Data
Jan. 26, 2011 (KR) 10-2011-0007908

(51) Int. Cl. A61B 8/00

(52) U.S. Cl. 600/443

(57) ABSTRACT

There are provided an ultrasound system with an opacity setting unit capable of setting opacity corresponding to rendering of volume data throughout depth. In one embodiment, an ultrasound system comprises an opacity setting unit configured to receive input information for setting opacity corresponding to rendering of volume data throughout the depth.

### CROSS-REFERENCE TO RELATED APPLICATION

The present application claims priority from Korean Patent Application No. 10-2011-0007908 filed on Jan. 26, 2011, the entire subject matter of which is incorporated herein by reference.

### TECHNICAL FIELD

The present disclosure generally relates to ultrasound systems, and more particularly to an ultrasound system with an opacity setting unit configured to set opacity for rendering volume data throughout depth.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two or three-dimensional ultrasound images of internal features of a target object (e.g., human organs).

The ultrasound system may provide a 3D ultrasound image including clinical information such as spatial information and anatomical figures of the target object, which cannot be provided by a 2D ultrasound image. The ultrasound system may transmit ultrasound signals into a target object and receive ultrasound echo signals reflected from the target object. The ultrasound system may further form volume data based on the ultrasound echo signals. The ultrasound system may also render the volume data to thereby form the 3D ultrasound image.

The ultrasound system may set opacity for rendering the volume data based on an intensity corresponding to each of the voxels of the volume data. Thus, it is required to the ultrasound system with an opacity setting unit capable of setting the opacity throughout depth.

### SUMMARY

There is provided an ultrasound system with an opacity setting unit capable of setting opacity corresponding to rendering of volume data throughout depth.

In one embodiment, by way of non-limiting example, an ultrasound system comprises an opacity setting unit configured to receive input information for setting opacity corresponding to rendering of volume data throughout the depth.

This Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit.

FIG. 3 is a schematic diagram showing an example of acquiring ultrasound data corresponding to a plurality of frames.

FIG. 4 is a schematic diagram showing an example of a user input unit.

FIG. 5 is a flow chart showing a process of setting opacity throughout depth.

FIG. 6 is a schematic diagram showing an example of volume data.

FIG. 7 is a schematic diagram showing an example of input information.

FIG. 8 is a schematic diagram showing an example of soft buttons.

### DETAILED DESCRIPTION

A detailed description may be provided with reference to the accompanying drawings. One of ordinary skill in the art may realize that the following description is illustrative only and is not in any way limiting. Other embodiments of the present invention may readily suggest themselves to such skilled persons having the benefit of this disclosure.

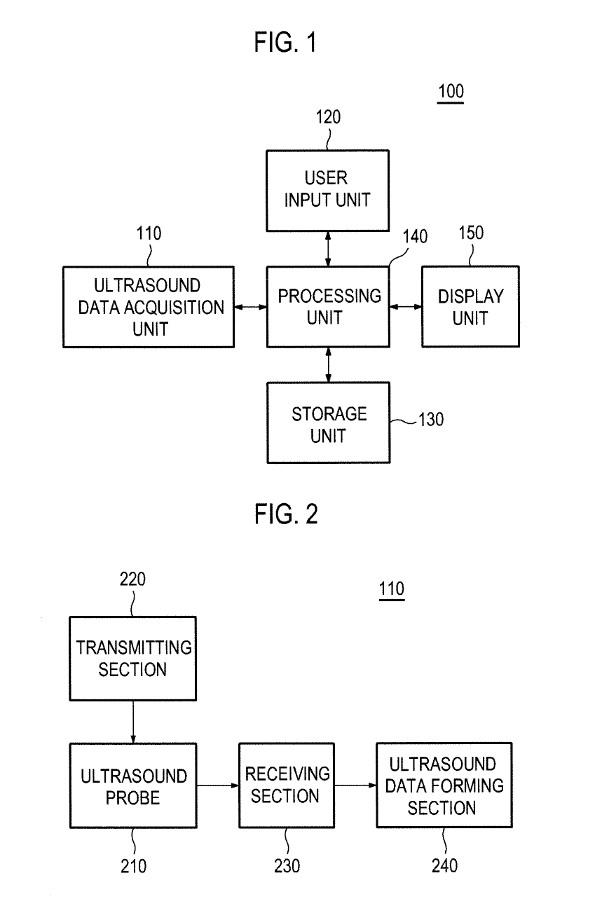

Referring to FIG. 1, an ultrasound system 100 in accordance with an illustrative embodiment is shown. As depicted therein, the ultrasound system 100 may include an ultrasound data acquisition unit 110.

The ultrasound data acquisition unit 110 may be configured to transmit ultrasound signals to a living body. The living body may include target objects (e.g., blood vessels, blood flow, a heart, a liver, etc.). The ultrasound data acquisition unit 110 may be further configured to receive ultrasound signals (i.e., ultrasound echo signals) from the living body to acquire ultrasound data.

FIG. 2 is a block diagram showing an illustrative embodiment of the ultrasound data acquisition unit. Referring to FIG. 2, the ultrasound data acquisition unit 110 may include an ultrasound probe 210.

The ultrasound probe 210 may include a plurality of transducer elements (not shown) for reciprocally converting between ultrasound signals and electrical signals. The ultrasound probe 210 may be configured to transmit the ultrasound signals to the living body. The ultrasound probe 210 may be further configured to receive the ultrasound echo signals from the living body to output received signals. The ultrasound probe 210 may include a three-dimensional mechanical probe, a two-dimensional array probe and the like.

The ultrasound data acquisition unit 110 may further include a transmitting section 220. The transmitting section 220 may be configured to control the transmission of the ultrasound signals. The transmitting section 220 may be further configured to generate electrical signals (“transmitting signals”) for obtaining an ultrasound image in consideration of the elements and focusing points. The transmitting section 220 may include a transmitting signal generating section (not shown), a transmitting delay time information memory (not shown), a transmitting beam former (not shown) and the like.

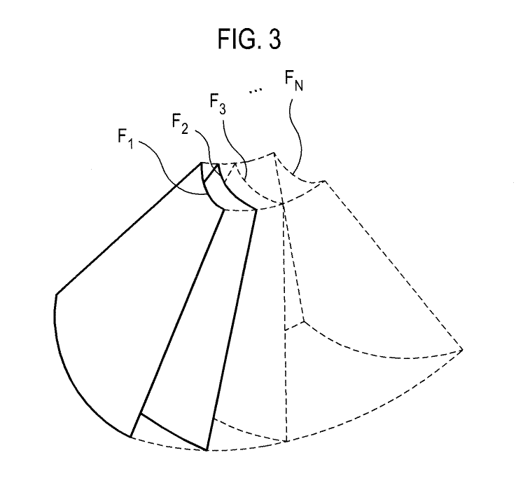

In the embodiment, the transmitting section 220 may form the transmitting signals for obtaining a plurality of frames Fᵢ (1≦i≦N) corresponding to a three-dimensional ultrasound image at every predetermined time, as shown in FIG. 3. Thus, the ultrasound probe 210 may convert the transmitting signals provided from the transmitting section 220 into the ultrasound signals, transmit the ultrasound signals to the living body and receive the ultrasound echo signals from the living body to thereby output the received signals.

FIG. 3 is a schematic diagram showing an example of acquiring ultrasound data corresponding to the plurality of frames Fᵢ (1≦i≦N). Referring to FIG. 3, the plurality of frames Fᵢ (1≦i≦N) may represent sectional planes of the living body (not shown). However, it should be noted herein that the plurality of frames Fᵢ (1≦i≦N) may not be limited thereto.

Referring back to FIG. 2, the ultrasound data acquisition unit 110 may further include a receiving section 230. The receiving section 230 may be configured to convert the received signals into digital signals. The receiving section 230 may be also configured to apply delays to the digital signals in consideration of the elements and the focusing points to thereby output digital receive-focused signals. The receiving section 230 may include an analog-to-digital converter (not shown), a receiving delay time information memory (not shown), a receiving beam forming (not shown) and the like.

The ultrasound data acquisition unit 110 may further include an ultrasound data forming section 240. The ultrasound data forming section 240 may be configured to form ultrasound data corresponding to the frames Fᵢ (1≦i≦N) based on the digital receive-focused signals provided from the receiving section 230. The ultrasound data may include radio frequency data. However, it should be noted herein that the ultrasound data may not be limited thereto. The ultrasound data forming section 240 may be also configured to perform signal processing (e.g., gain control, etc) upon the digital receive-focused signals.

Referring back to FIG. 1, the ultrasound system 100 may further include a user input unit 120. The user input unit 120 may be configured to receive input information of a user. In the embodiment, the input information may include first input information for selecting a diagnostic mode corresponding to the three-dimensional ultrasound image. The input information may further include second input information for setting opacity corresponding to rendering of the volume data throughout depth. The depth may represent depth in a rendering direction. However, it should be noted herein that the depth may not be limited thereto.

In the embodiment, the user input unit 120 may include an opacity setting unit configured to receive the second input information for setting the opacity throughout the depth.

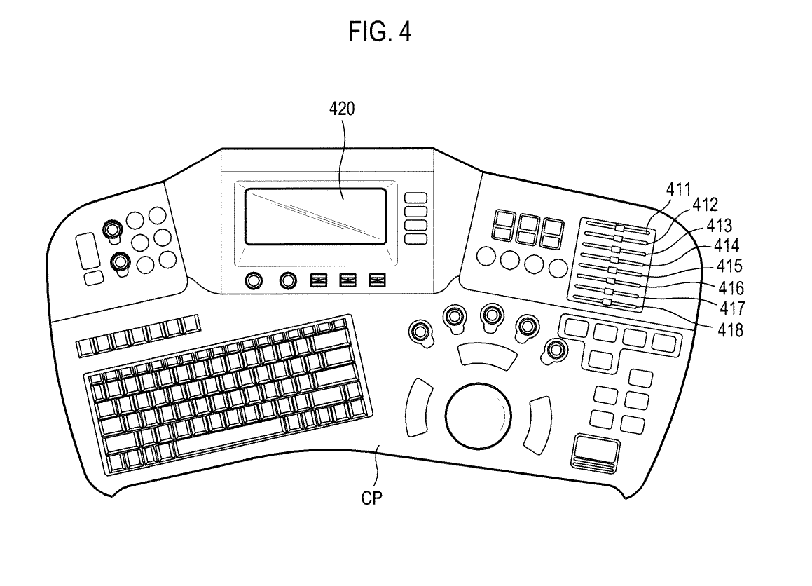

As one example, the opacity setting unit may include a plurality of time gain compensation sliders 411 to 418 of a control panel CP, as shown in FIG. 4. The time gain compensation sliders 411 to 418 may set the opacity of 0 to 255.

As another example, the opacity setting unit may include a plurality of soft buttons 811 to 818, which are displayed on a touch screen 420 of the control panel CP, as shown in FIG. 8. The soft buttons 811 to 818 may set the opacity of 0 to 255.

Although it has been described that the soft buttons 811 to 818 may be displayed on the touch screen 420 of the control panel CP, the soft buttons 811 to 818 may be further displayed on a display unit 150 corresponding to the touch screen.

The ultrasound system 100 may further include a storage unit 130. The storage unit 130 may store the ultrasound data acquired by the ultrasound data acquisition unit 110. The storage unit 130 may further store a mapping table for providing the depth and an opacity setting range corresponding to the opacity setting unit of the user input unit 120. For example, the storage unit 130 may store the mapping table as shown in Table 1.

TABLE 1

| | | Opacity setting |
|---|---|---|
| Opacity setting unit | Depth | range |
| Time gain compensation slider 411 | 0~2 cm | 0~255 |
| or soft button 811 | | |
| Time gain compensation slider 412 | 2.1 cm~4.0 cm | 0~255 |
| or soft button 812 | | |
| Time gain compensation slider 413 | 4.1 cm~6.0 cm | 0~255 |
| or soft button 813 | | |
| Time gain compensation slider 414 | 6.1 cm~8.0 cm | 0~255 |
| or soft button 814 | | |
| Time gain compensation slider 415 | 8.1 cm~10.0 cm | 0~255 |
| or soft button 815 | | |
| Time gain compensation slider 416 | 10.1 cm~12.0 cm | 0~255 |
| or soft button 816 | | |
| Time gain compensation slider 417 | 12.1 cm~14.0 cm | 0~255 |
| or soft button 817 | | |
| Time gain compensation slider 418 | 14.1 cm~16.0 cm | 0~255 |
| or soft button 818 | | |

The ultrasound system 100 may further include a processing unit 140. The processing unit 140 is in communication with the ultrasound data acquisition unit 110, the user input unit 120 and the storage unit 130. The processing unit 140 may include a central processing unit, a microprocessor, a graphic processing unit and the like.

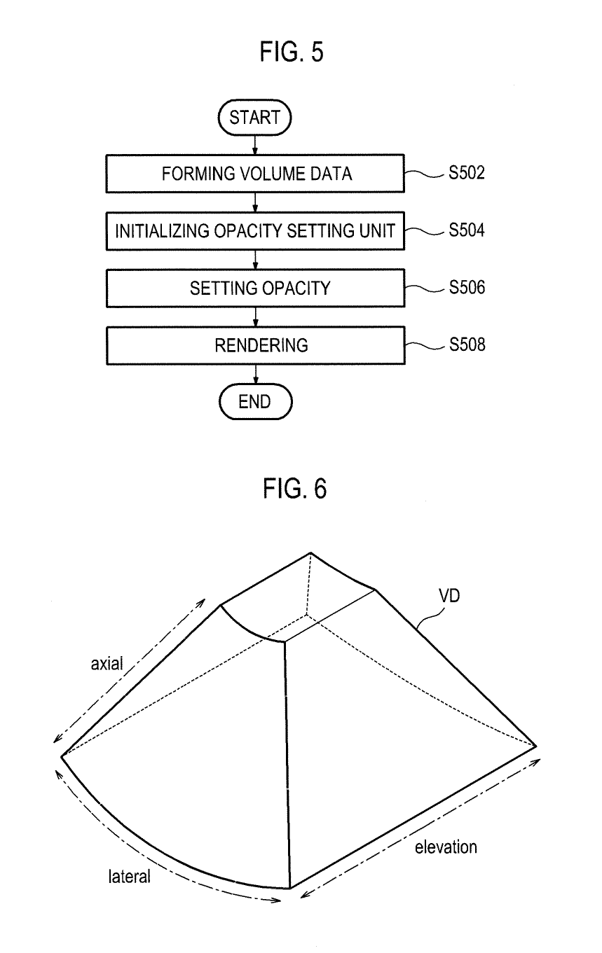

FIG. 5 is a flow chart showing a process of forming the three-dimensional ultrasound image. Referring to HG 5, the processing unit 140 may be configured to form volume data VD based on the input information (i.e., first input information) provided from the user input unit 120 as shown in FIG. 6, at step S502 in FIG. 5. The volume data VD may be formed by using the ultrasound data provided from the ultrasound data. The volume data may be stored in the storage unit 130.

FIG. 6 is a schematic diagram showing an example of the volume data VD. The volume data VD may include a plurality of voxels (not shown) having brightness values. In FIG. 6, the axial direction may be a transmission direction of the ultrasound signals, the lateral direction may be a longitudinal direction of the elements, and the elevation direction may be a swing direction of the elements, i.e., a depth direction of the 3D ultrasound image.

Referring back to FIG. 5, the processing unit 140 may be configured to initialize the opacity setting unit of the user input unit 120 based on the first input information, at step S504 in FIG. 5.

As one example, the processing unit 140 may convert the plurality of time gain compensation sliders 411 to 418 of the control panel CP into the opacity setting unit based on the first input information. The processing unit 140 may further set the opacity of 0 to 255 on each of the time gain compensation sliders 411 to 418.

As another example, the processing unit 140 may form the plurality of soft buttons 811 to 818. The processing unit 140 may further set the opacity of 0 to 255 on each of the soft buttons 811 to 818. The processing unit 140 may further control display of the soft buttons 811 to 818.

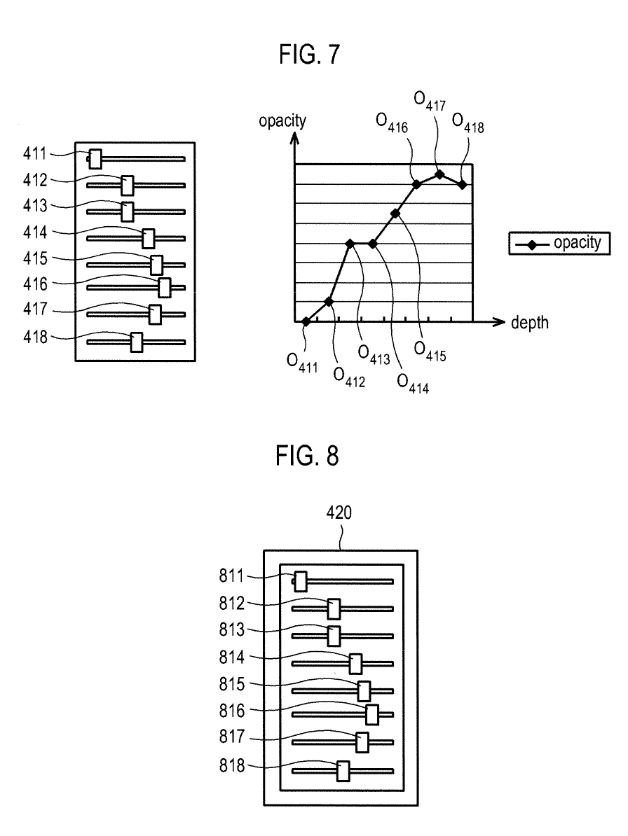

The processing unit 140 may be configured to set the opacity corresponding to the input information (i.e., second input information) provided from the user input unit 120 based on the mapping table, at step S506 in FIG. 5. For example, the processing unit 140 may set the opacity O₄₁₁ to O₄₁₈ corresponding to the second input information based on the mapping table, as shown in FIG. 7. The opacity O₄₁₁ to O₄₁₈ may correspond to the time gain compensation sliders 411 to 418, respectively.

The processing unit 140 may be configured to render the volume data VD based on the set opacity to thereby form the three-dimensional ultrasound image, at step S508 in FIG. 5. The methods of rendering volume data based on the opacity are well known in the art. Thus, they have not been described in detail so as not to unnecessarily obscure the present disclosure.

Referring back to FIG. 1, the ultrasound system 100 may further include the display unit 150. The display unit 150 may display the three-dimensional ultrasound image formed by the processing unit 140. The display unit 150 may further display the soft buttons 811 to 818.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
an opacity setting unit configured to receive input information for setting opacity corresponding to rendering of volume data throughout the depth.

2. The ultrasound system of claim 1, further comprising:
an ultrasound data acquisition unit configured to transmit ultrasound signals to a living body and receive ultrasound echo signals from the living body to acquire ultrasound data;
a storage unit configured to store a mapping table for providing depth and an opacity setting range corresponding to the opacity setting unit; and
a processing unit configured to form the volume data based on the ultrasound data, set the opacity corresponding to the input information based on the mapping table, and render the volume data based on the opacity.

3. The ultrasound system of claim 1, wherein the opacity setting unit includes a plurality of time gain control sliders.

4. The ultrasound system of claim 1, wherein the opacity setting unit includes a plurality of soft buttons.
